(19) United States
(12) Patent Application Publication
(10) Pub. No.: US 2012/0046683 A1
(43) Pub. Date: Feb. 23, 2012

(54) DEVICES AND METHODS FOR TEMPORARILY OPENING A BLOOD VESSEL

(76) Inventors: Scott Wilson, Redwood City, CA (US); Kirk Pedersen, San Francisco, CA (US); Ryan Grandfield, Livermore, CA (US); Emily Vu, San Jose, CA (US); John Miller, Redwood City, CA (US)

(21) Appl. No.: 13/282,380

(22) Filed: Oct. 26, 2011

Related U.S. Application Data
(63) Continuation of application No. 12/290,646, filed on Oct. 31, 2008.

(51) Int. Cl. A61M 29/00

(52) U.S. Cl. 606/194

(57) ABSTRACT

A device for temporarily opening a blood vessel is provided. The device has an expandable cage mounted over a shaft having a lumen. The lumen has a distal end, which extends beyond the distal end of the cage so that the lumen provides access to the vasculature distal to the obstruction.

### BACKGROUND OF THE INVENTION

The present invention is directed to devices and methods for temporarily opening a blood vessel. The present invention may be used to temporarily open a blood vessel in any part of the body such as in the cerebral vasculature.

The present invention is also directed to opening blood vessels and may be useful in dissolving or removing obstructions from blood vessels as well. Mechanical removal of obstructions can be problematic at times due to the forces necessary to release the obstruction from the vessel wall. This could be a result of the obstruction physiologically binding to the vessel wall, increased blood pressure at the face of the clot, vessel collapse during retrieval and most likely some combination of any or all of these scenarios.

### SUMMARY OF THE INVENTION

The present invention provides a cage, which is used to temporarily open a vessel. In one application, the cage may be used to open a vessel at a location where an obstruction exists. The cage is positioned within the obstruction and expanded to enhance blood flow in the region. The cage has a relatively open structure, which permits blood to pass therethrough. As such, the natural dissolution of the obstruction can be accelerated or enhanced by providing enhanced blood flow through the region.

Another advantage of the present invention is that the enhanced blood flow through the obstruction may help to perfuse blood to the oxygen-starved tissue distal to the occlusion thereby minimizing or eliminating tissue necrosis and severity of the stroke.

After a period of time, the obstruction is assessed using an angiogram or other method of determining to what degree the obstruction has been dissolved. An obstruction-removing device may also be delivered through the lumen to engage a distal portion of the obstruction. The cage may also be used to remove the obstruction. The cage may remain expanded or may be partially collapsed when aiding in removal of the obstruction. The removal device and cage may be used together to remove the obstruction by simultaneously manipulating the cage and removal device.

In another aspect of the present invention, the cage may form a reinforcing portion of the shaft, which extends proximal to the cage and/or distal to the cage. Using the cage to reinforce the catheter shaft may help to reduce the overall profile of the catheter.

These and other aspects of the present invention will become apparent from the following description of the preferred embodiments.

### BRIEF DESCRIPTION OF DRAWINGS

FIG. 1 shows a catheter in accordance with the present invention.

FIG. 2 shows the catheter positioned within an obstruction.

FIG. 3 shows a cage expanded within the obstruction.

FIG. 4 shows a portion of the obstruction, which has dissolved.

FIG. 5 shows a removal device used with the catheter.

FIG. 6 shows the cage deployed within the vessel.

FIG. 7 shows a snare used to re-engage the cage.

FIG. 8 shows the snare tightened around the shaft.

FIG. 9 shows another catheter advanced over the snare.

FIG. 10 shows another catheter having an expandable cage.

FIG. 11 shows the catheter expanded within an obstruction.

### DESCRIPTION OF THE PREFERRED EMBODIMENTS

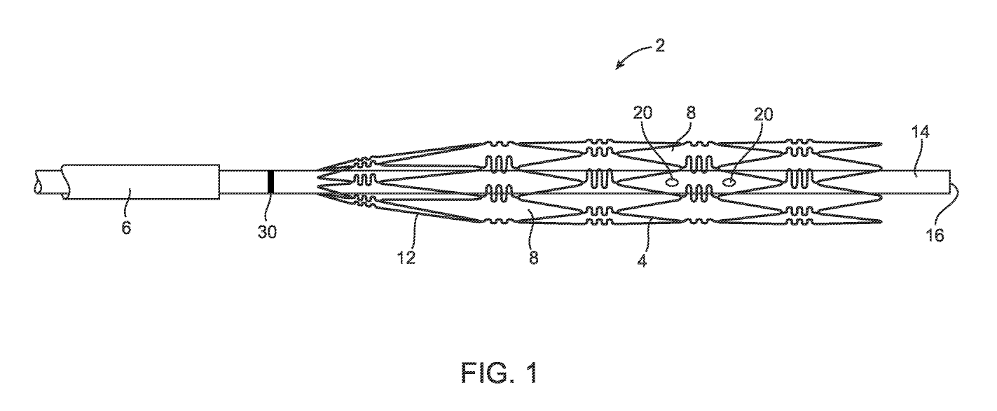

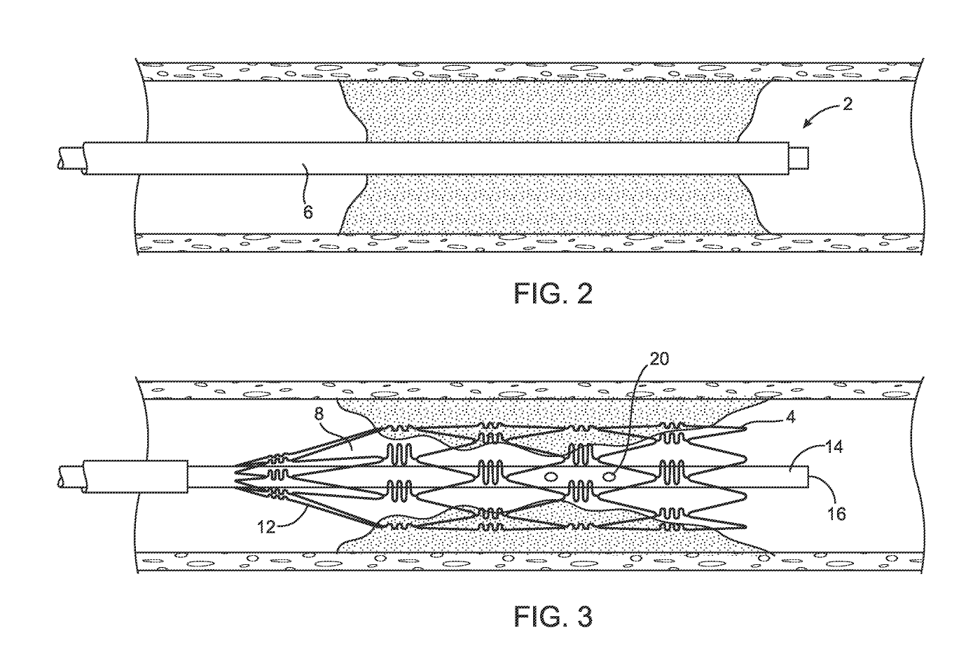

Referring to FIGS. 1-3, a device 2 for temporarily opening a blood vessel is shown. The device 2 includes an expandable cage 4. The cage 4 is naturally biased toward the expanded position of FIG. 1 but can be collapsed into a delivery catheter 6 as shown in FIG. 2. The cage 4 may also be expanded with a balloon or the like rather than being self-expandable without departing from various aspects of the present invention.

The cage 4 may be formed in a manner similar to a stent. As such, the cage 4 may be formed from a single integrally formed piece of material such as a stainless steel or nitinol tube with material removed from the tubes to form openings 8 in the cage 4. The cage 4 may also be formed in any other manner, which produces a relatively open structure for the reasons described below. The cage 4 has a relatively open structure so that the openings 8 in a proximal portion 12 of the cage 4 may permit blood or other fluids to pass therethrough.

The cage 4 is mounted over a shaft 14 having a lumen 16. The shaft 14 extends beyond a distal end 18 of the cage 4 so that the lumen 16 may be used to access the vasculature distal to the cage 4 as described below. The lumen 16 may also have one or more holes 20 along the portion of the lumen 16 positioned beneath the cage 4 to deliver therapeutics. The hole(s) 20 may enhance blood flow in this area to help dissolve the obstruction as explained below.

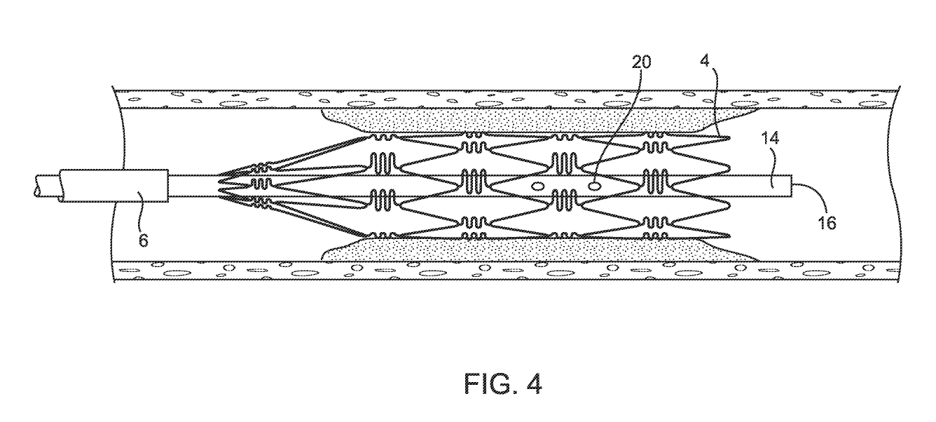

The present invention may be useful in dissolving or removing obstructions from blood vessels. To this end, the cage 4 is advanced to a location where an obstruction is blocking blood flow in a vessel. The delivery catheter 6 is advanced through the obstruction as shown in FIG. 2 and then withdrawn to expose the cage 4 as shown in FIG. 3. The delivery catheter 6 may be the catheter described in U.S. patent application Ser. No. 11,490,843, filed Jul. 21, 2006, which is incorporated herein by reference. The cage 4 will automatically expand within the vessel to partially open the vessel as shown in FIG. 3. The openings 8 in the proximal portion of the cage 4 may permit some blood to pass through the obstruction. As such, the natural dissolution of obstruction can be accelerated or enhanced by providing some blood flow across the obstruction as shown in FIG. 4. Another advantage of the present invention is that the enhanced blood flow through the obstruction may help to perfuse blood to the oxygen-starved tissue distal to the occlusion thereby minimizing or eliminating tissue necrosis and severity of the stroke. As described herein, the methods of temporarily opening a blood vessel include, of course, the concept of opening an obstruction, which lies within the blood vessel thereby opening the vessel as well.

The cage 4 may be maintained in the blood vessel for a period of time to achieve some of the benefits described above. After a period of time, which may be as little as a few minutes or as long as several days, an angiogram (or other visualization method such as CT, MRI or Ultrasound, etc.) can be performed to assess the status of the obstruction. If the clot has dissolved and flow has been restored, the cage can be collapsed using the delivery catheter 6 and then removed. If the clot has not fully dissolved in a satisfactory time period, the obstruction may be removed mechanically.

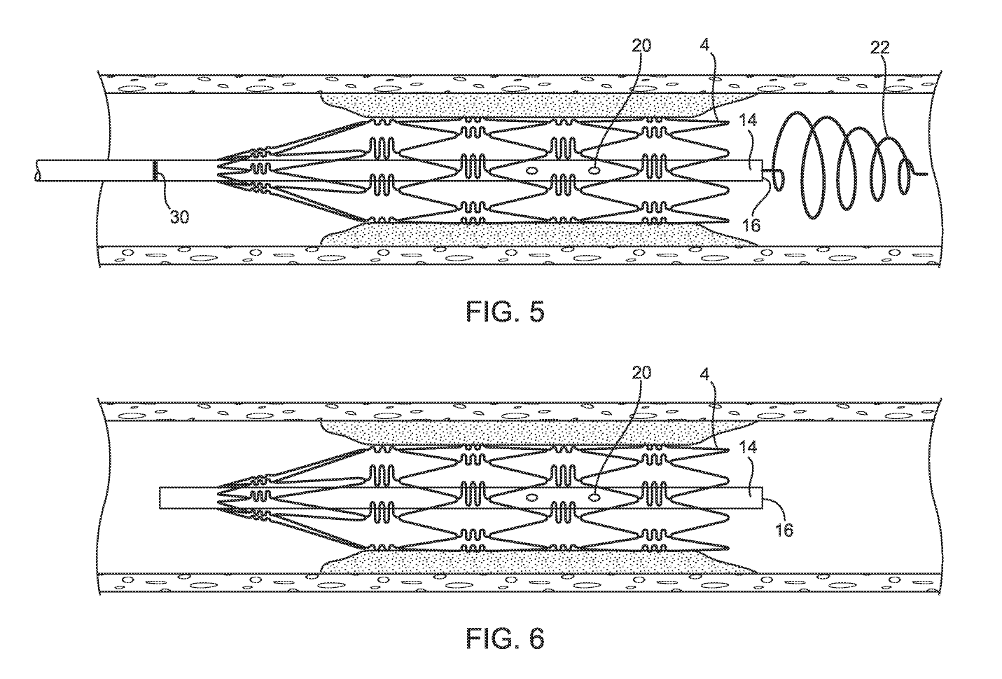

Referring to FIG. 5, an obstruction removal device 22 is shown which may be used to remove the obstruction. The device 22 is advanced through the lumen 16 until the device 2 extends from the distal end of the lumen 16. The removal device 22 may then be manipulated to engage and ensnare the obstruction. For example, the device 22 may be twisted in one, both or neither direction and pulled proximally to engage and ensnare the obstruction. The obstruction removal device 22 may take any other suitable form without departing from numerous aspects of the present invention.

The cage 4 may also be used to help remove the obstruction. The cage 4 may be left partially (or even fully) expanded to help aid in removing the obstruction. The cage 4 may be manipulated independently of the removal device 22 or may be manipulated with the removal device 22. For example, the cage 4 and removal device 22 may be moved proximally together to dislodge and remove the obstruction.

Referring to FIG. 6, the cage 4 may also be deployed within the blood vessel. The cage 4 may be released using any suitable connection such as an electrolytically severable connection 30 (see FIG. 1) as is known in the art. The cage 4 is then left in the body to achieve some of the benefits described herein. The cage 4 may then be re-engaged and removed as now described.

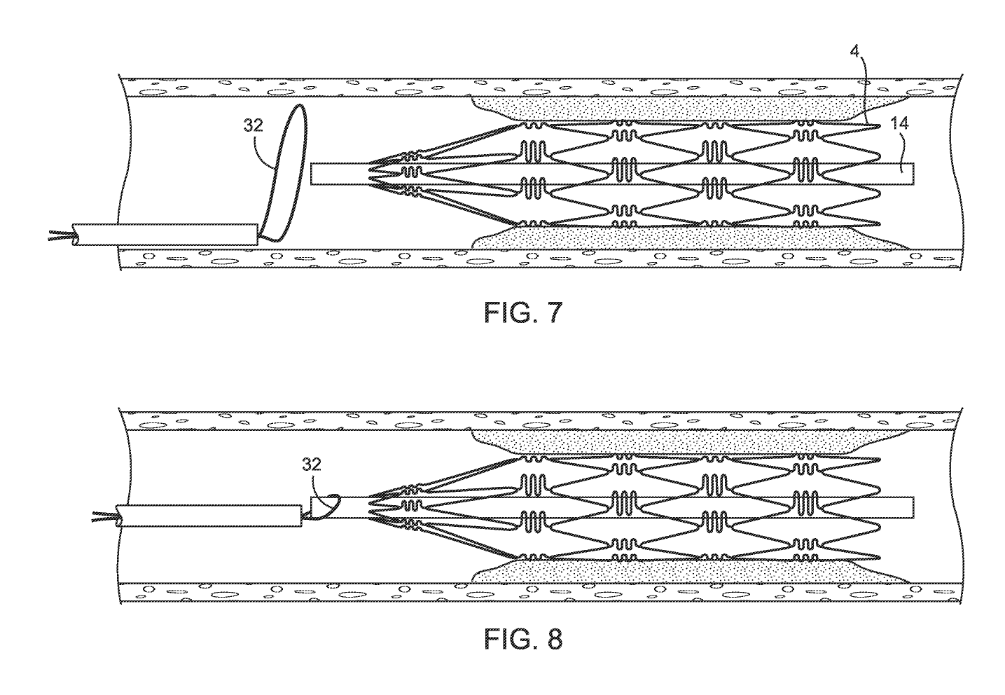

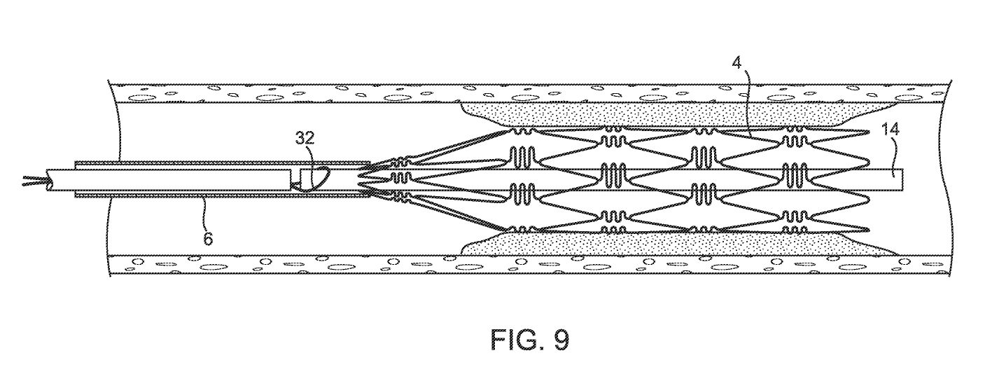

A snare 32 is deployed through the delivery catheter 6 to re-engage the cage 4 as shown in FIGS. 7-9. The snare 32 is moved over a proximal portion of the catheter 14 and closed to hold the catheter 14. The catheter 6 is then advanced while maintaining tension on the snare 32 to prevent the cage 4 from moving as the catheter 6 is advanced. As the catheter 6 is advanced, the cage 4 is collapsed into the catheter 6 for removal. Once the catheter 6 has become re-engaged with the cage 4, the removal device 22 may also be used to help remove the obstruction if necessary. The cage 4 may be re-engaged in any other suitable manner other than a snare 32 such as a catheter or wire having a hook or a mechanical connector.

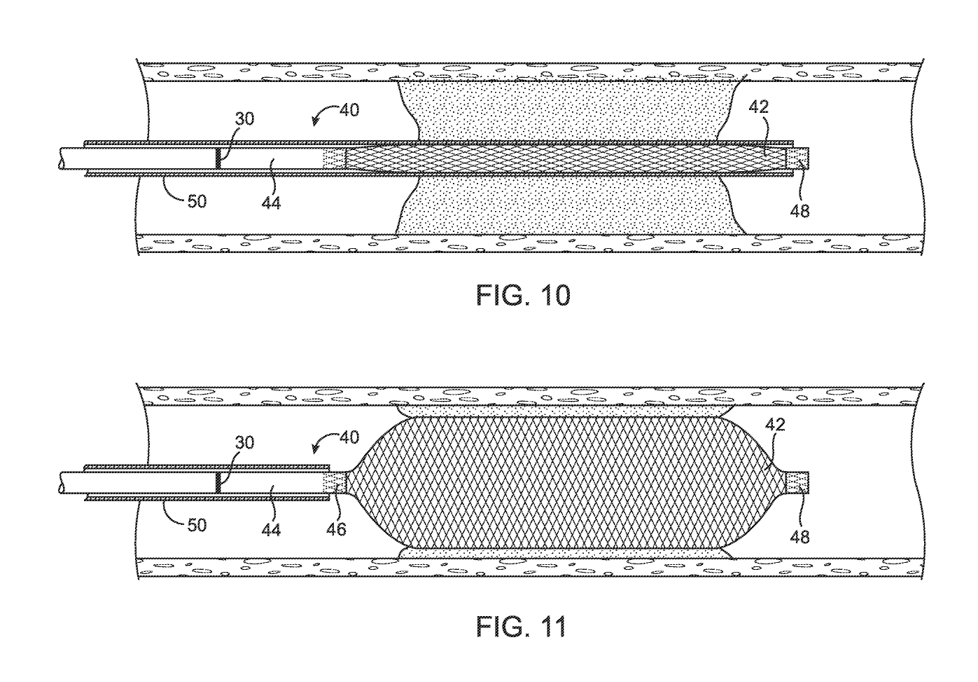

Referring to FIGS. 10 and 11, another catheter 40 is shown which has a cage 42 to temporarily open a blood vessel. The catheter 40 includes a shaft 44 that is reinforced by the cage 42. The cage 42 may extend into and reinforce a proximal shaft portion 46 and/or a distal shaft portion 48 which extend from the proximal and distal ends of the cage 42. The cage 42 is a braided structure but may also be a helical coil, or an integrally formed stent-like structure without departing from various aspects of the invention. The cage 42 will automatically expand when moved outside a delivery catheter 50 as shown in FIG. 11. Using the cage to reinforce the catheter shaft may help to reduce the overall profile of the catheter 40.

The catheter 40 may be used in the same manner as the other catheters described herein and such use is expressly incorporated here. For example, the catheter 40 may include the electrolytically severable connection 30 so that the cage 42 and a portion of the shaft 44 may be released within the vessel. The catheter 40 may also be used to remove the obstruction as mentioned above together with or independent of the removal device 22 (FIG. 5).

1-8. (canceled)

9. A device for temporarily opening a blood vessel, comprising:
a shaft having a lumen with a distal end; and
an expandable cage having openings therein, the cage being coupled to the shaft, the cage being mounted over the shaft and being movable from a collapsed position to an expanded position, the shaft extending beyond the distal end of the cage when the cage is in the expanded position.

10. The device of claim 9, wherein:
the lumen in the shaft has at least one hole therein in a portion of the shaft lying beneath the cage.

11. The device of claim 9, wherein:
the expandable cage is separable from the shaft so that the cage may be deployed.

12. The device of claim 9, wherein:
the shaft is configured to re-engage the cage after the cage has been deployed, the shaft being configured to collapse the cage from the expanded position to the collapsed position as the shaft is advanced over the cage.

13. A method for temporarily opening a blood vessel, comprising:
providing an expandable cage having openings therein, the cage being coupled to a shaft having a lumen, the cage being movable from a collapsed position to an expanded position, the cage having a proximal end and a distal end, the cage forming a reinforcing portion of the shaft which extends proximally from the proximal end of the cage;
advancing the cage through a patient's vascular system;
expanding the cage to temporarily open a portion of a blood vessel, the cage having an open shape which may permit blood to pass therethrough; and
removing the cage after a period of time.

14. The method of claim 13, wherein:
the providing step is carried out with the shaft having a distal shaft portion, the distal shaft portion extending distally from the cage, the cage forming a reinforcing portion of the distal shaft portion.

15. The method of claim 13, further comprising the step of:
releasing the cage so that the cage remains within the patient.

16. The method of claim 15, further comprising the step of:
coupling a catheter to the cage after the releasing step.

17. A device for temporarily opening a blood vessel, comprising:
a shaft having a lumen with a distal end; and
an expandable cage having openings therein, the cage being coupled to the shaft, the cage being movable from a collapsed position to an expanded position, the cage having a proximal end and a distal end, the cage forming a reinforcing portion of the shaft which extends proximally from the proximal end of the cage.

18. The device of claim 17, wherein:
the shaft has a distal shaft portion, the distal shaft portion extending distally from the cage, the cage forming a reinforcing portion of the distal shaft portion.
